(19) United States
(12) Patent Application Publication
(10) Pub. No.: US 2005/0065411 A1
(43) Pub. Date: Mar. 24, 2005

(54) Tongue depressing device

(76) Inventors: Blair F. Baldwin, Newport, RI (US); Darius J. Nasiek, Wayne, NJ (US); Blair F. Baldwin JR., Somerville, MA (US); W. Barton Baldwin, Newport, RI (US)

Correspondence Address: ROPES & GRAY LLP, ONE INTERNATIONAL PLACE, BOSTON, MA, 02110-2624, US

(21) Appl. No.: 10/941,278

(22) Filed: Sep. 14, 2004

Related U.S. Application Data
(60) Provisional application No. 60/502,971, filed on Sep. 15, 2003.

(51) Int. Cl. A61B 1/32

(52) U.S. Cl. 600/240

(57) ABSTRACT

An S-shaped tongue depressing device which configured in such a manner that it conforms to not only the transverse contour of the tongue, but also longitudinal shape of the tongue.

### RELATED APPLICATION

This application claims the benefit of U.S. Provisional application 60/502,971, entitled Tongue Depressing Device and filed Sep. 15, 2003, naming Darius Jacek Nasiek, Blair Fahnestock Baldwin, and Blair Fahnestock Baldwin, Jr. as inventors. The entire specification and teachings of the referenced provisional application are expressly incorporated herein by reference.

### BACKGROUND OF THE INVENTION

Historically, the commonly available and used device for depressing the tongue by a medical or health professional, in order to enable the professional to examine a patient's mouth and/or throat, has been a thin rectangular linear wooden strip of natural color and with rounded edges. This tongue depressing device or tongue depressor typically does not bear a laminate covering. Such a tongue depressing device is not user friendly, for either the patient or the medical professional. For example, neither end of the device is designed to take into account the feel or needs of the patient, who not infrequently experiences gagging or choking, or the medical professional, who must manage to conduct an oral examination, despite the fact that the view of the mouth and throat may be obstructed even when the tongue and lips are depressed or moved as far as possible. In addition, the commonly used tongue depressing device is typically available in only one size, despite the wide range of patient sizes. Further, the traditional tongue depressing device is not aesthetically pleasing to patients, such as young children who may have had unpleasant experiences during oral examinations. There is a longstanding need for a tongue depressing device which can not only be used easily and effectively by a medical professional, but also be comfortable and even aesthetically pleasing to patients.

### SUMMARY OF THE INVENTION

Described herein is a tongue depressing device, also referred to as a tongue depressor, which is configured in such a manner that it conforms to not only the transverse contour (the shape or curve along the short axis) of the tongue, but also the longitudinal contour (the shape or curve along the long axis) of the tongue. The tongue depressing device of the present invention is easy to properly insert into an individual's mouth or oral cavity, as well as to use to depress, move, hold or immobilize the tongue, with less likelihood that the individual will gag or choke than is the case with other available tongue depressing devices, such as the often used wooden tongue depressor. Further, because of its configuration, the tongue depressing device fits over the tongue and lowers the angle at which it is held by the user, such as a health care provider, with the result that the device does not interfere with the ability of the user to see into the mouth and visualize the throat and epiglottis. The configuration also permits the user to securely hold the tongue so that it does not interfere with visualization of the oral cavity and epiglottis, and to manipulate its position.

As further described and illustrated herein, when viewed from the side, the tongue depressing device of the present invention is an ergonomically designed S-shaped, channeled device whose configuration is such that when the device is in use, the user's hand (e.g., the hand of a health care professional) is sufficiently below the mouth of the individual being examined that the user can view the mouth and/or throat with ease. The tongue depressing device can be any of a variety of shapes, including rectangular (with or without rounded corners or ends), elliptical or oval, or hour glass shaped. The tongue depressing device, viewed from the top, can also be any of a variety of imaginative shapes, such as a cartoon or other character (e.g., a mermaid, fish), an animal, plant, or other form appropriate for individuals (particularly children) who will be examined. Either or both surfaces of the tongue depressing device can be smooth and continuous (uninterrupted) or it can comprise smooth areas, textured areas (e.g., which help hold the tongue to the undersurface of the depressing device) and/or openings, surface gripping channels or protrusions, which also help hold the tongue in position. The device can be solid or can comprise a space between the upper and lower surfaces, provided that the device is sufficiently rigid to be used for its intended purposes. For example, the tongue depressing device can be comprised of an upper and a lower surface, with a space or open area between. For example, a tongue depressing device of this invention can comprise an upper surface and a lower surface which are joined together or continuous at a sufficient number of points or sections along their edges and/or respective inner surfaces that they form the desired S-shaped, channeled configuration. Alternatively, there can be one or more openings of any shape in the device, as described further below.

The tongue depressing device is made of any of a wide variety of materials which are sufficiently stiff/rigid and resilient and suitable for introduction into the mouth, such as non-toxic thermoplastic material. These include, but are not limited to, any food grade plastic or FDA approved/approvable plastic or other material, such as polystyrene (e.g., high impact polystyrene), polyethylene, polypropylene, polyvinyl chloride or a metal, such as an autoclavable metal (e.g., medical grade aluminum). Whatever the material used, the tongue depressing device can be made by any of a variety of methods known to those in the art, such as, but not limited to, injection molding, extrusion processing or being cut or stamped from a sheet of the selected material.

The tongue depressing device can include a variety of additional features and characteristics which will make it particularly well suited for specific uses. For example, markings can be included on one or more surfaces of the device, thus making it useful as a measuring instrument (e.g., for determining the size of lesions on an individual's tongue, inside the mouth or at the beginning of the throat) and avoiding the need for a separate measuring tool. In addition, for example, the tongue depressing device can have affixed to a surface a compartment containing separated components which, when mixed, produce a light source, thus making it easier for the user to see inside the mouth. The tongue depressing device can also have incorporated within it or coated thereon any one or more of numerous materials which enhance its utility. For example, antipathogenic materials, such as antibacterial, antiviral or antifungal agents (drugs) can be used, such as by incorporating such an agent or agents into the material from which the depressing device will be produced, impregnating the tongue depressing device with such agent(s) or coating, depositing or otherwise adding such agent(s) to the surface of the formed tongue depressing device. Such agents are useful to reduce or prevent bad breath in the individual being examined. Other materials, such as flavorings, colorings and scents, can also be incorporated into the depressing device as it is produced, or added to the surface of the resulting device. Anesthesia, such as a topical anesthesia useful to temporarily numb or lessen the sensitivity of the tongue, can be present in (e.g., by impregnating it into the device body) or on the surface of the tongue depressing device. If desired, such as for pediatric uses or for educational or informational purposes, a design (e.g., a cartoon or other familiar character or image), message (e.g., popular sayings, medical message) or other printed material (e.g., dietary or other health related information) can be included on the surface of the tongue depressing device. Health related information or a medical message can also be included on the surface(s) of the tongue depressing device. The device can bear text; medically related shapes, designs or patterns and/or information which relates to any condition or disease, such as information which relates directly or indirectly to symptoms, afflictions, diseases or exams of the throat or oral cavity. These can be present on all or part of the tongue depressing device, such as on the midsection. In a further embodiment, the tongue depressing device of this invention includes a porous section, which typically is added to the surface of the device after it is formed and is useful for obtaining samples from the oral cavity, such as samples to be analyzed for bacteria, viruses or other pathogens or abnormal cells.

The tongue depressing device of this invention can be of one size, such as of a length and width similar to that of the well-known wooden tongue depressor, or can produced to be of a size appropriate for individuals of varying size (e.g., a pediatric size and adult size).

The subject tongue depressing device is not only patient friendly and convenient for use, but also useful for tasks that may not be conveniently carried out using currently available tongue depressing devices, such as the wooden tongue depressor so commonly used.

### BRIEF DESCRIPTION OF THE DRAWINGS

Various other objects, advantages and features of the subject invention will become apparent to those skilled in the art from the following discussion, taken in conjunction with the following drawings, in which:

FIG. 1 is a top view of a tongue depressor of the present invention.

FIG. 2 is a side view of a tongue depressor of the present invention.

FIG. 3 is a perspective view of a tongue depressor of the present invention.

FIG. 4 is a second perspective view of a tongue depressor of the present invention.

FIG. 5 is a further perspective view of a tongue depressor of the present invention.

FIG. 6 is a transverse cross section of a tongue depressor of the present invention, as viewed along axis A.

FIG. 7 is a longitudinal cross section of a tongue depressor of the present invention, as viewed along axis B.

### DETAILED DESCRIPTION OF THE INVENTION

Detailed embodiments of the present invention are disclosed herein. However, it is to be understood that the disclosed embodiments are only exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

The tongue depressing device of the present invention is now described with reference to the drawings, wherein like characteristics and features of the invention are designated by the same reference numbers or letters.

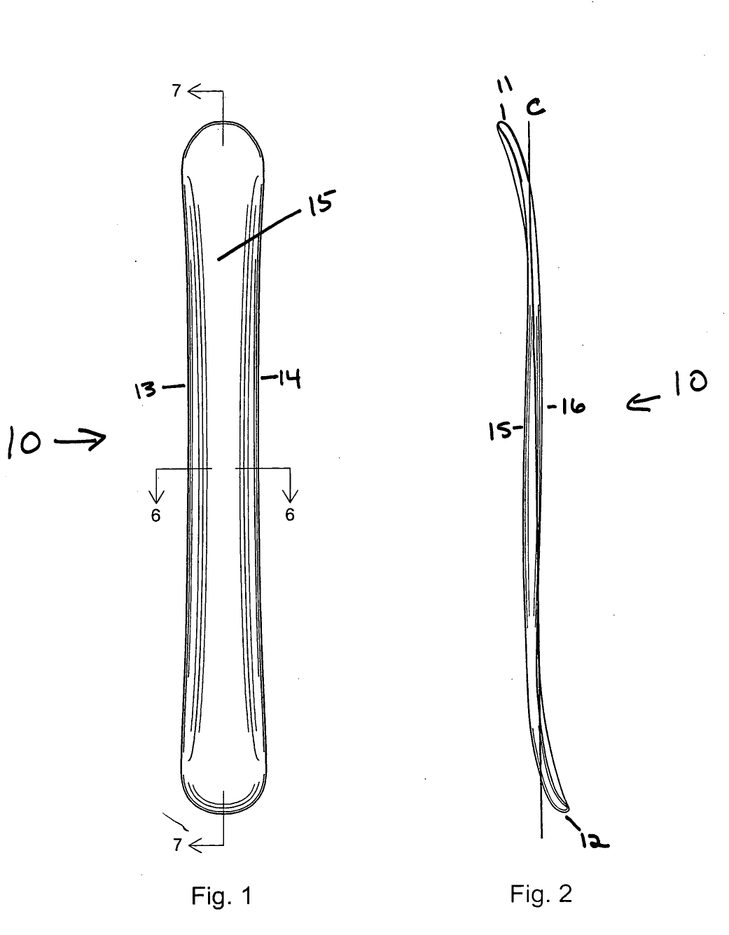

Referring to FIGS. 1-7, a tongue depressing device 10 is disclosed. As illustrated, tongue depressing device 10 is long and slender. In various embodiments of the tongue depressing device 10, the size (length, width, thickness) will differ and will be determined with reference to the age and/or size of individual with which tongue depressing device 10 will be used. In certain embodiments, tongue depressing device 10 is similar in length to the widely used wooden tongue depressor. The width of the device can be varied as needed and, in certain embodiments, is similar to that of the wooden tongue depressor. Specific embodiments of the device of this invention are a pediatric tongue depressing device and an adult tongue depressing device, which will be of different lengths and/or different widths, (e.g., the pediatric model may be shorter, narrower or both shorter and narrower than the adult model).

Surfaces 15 and 16, which are, respectively, the upper and lower surfaces of tongue depressing device 10, may be smooth along their entire length and width or may be a combination of smooth and textured areas. For example, a section of either or both of surfaces 15 and 16 may be textured, slightly roughened or uneven, in order to facilitate, for example, holding of the tongue or removal of materials (e.g., cells, fluids) from the tongue surface. As described further below, materials may be added to the surface for a variety of uses. Further, surfaces 15 and 16 can comprise protrusions and/or indentations, which can be introduced at the time tongue depressing device 10 is produced, such as by injection molding, or after it has been formed, such as by addition of one or more protrusions or removal of one or more sections of a surface to produce an indentation. The indentation can be such that it is partial or results in removal of a segment of both surfaces and the material therebetween (resulting in a hole in the device). Tongue depressing device 10 is relatively thin and can be of uniform or varying thickness. FIG. 1 is a top view of tongue depressing device 10, which illustrates relative measurements or proportions; in this embodiment, ends 11 and 12 of the device are round. In an alternative embodiment, tongue depressing device is as represented in FIG. 1 except that ends 11 and 12 are not rounded.

FIG. 2 is a side view of tongue depressing device 10, which illustrates its slight S configuration. As illustrated, ends 11 and 12 (which in this embodiment are rounded) are curved in opposite directions from one another. The curve of end 11 and end 12 is sufficient to fit over the tongue of the individual being examined and result in positioning of the user's hand such that tongue depressing device 10 does not interfere with visualization of the mouth, epiglottis or throat. The arc of the curves of ends 11 and 12 can be varied as needed and will be less than 90 degrees measured from line C. End 11 and end 12 can be flat or curved along the transverse axis. With reference to FIG. 2, the tongue depressing device 10 can be introduced into the mouth with the concave portion of surface 15 placed on the tongue (such that rounded end 11 is in the mouth) and rounded end 12 held by the user (e.g., a health care provider). Alternatively, again with reference to FIG. 2, the tongue depressing device 10 can be introduced into the mouth with the concave portion of surface 16 placed on the tongue (such that rounded end 12 is in the mouth) and rounded end 11 held by the user. Either end 11 or end 12 can be placed in the mouth or held by the user. When positioned in an individual's mouth, the angle of the user's hand is such that neither it nor tongue depressing device 10 obstructs the view into the mouth or throat to any significant extent. When tongue depressing device 10 is placed on the tongue of an individual, the convex surface of the end in the individual's mouth is on the top side. The midsection of tongue depressing device 10 (the section between the ends) can be substantially flat or slightly curved along the axis B. The S configuration makes it possible to stack or nestle together a number of the devices of the present invention, making it possible for them to be compactly stored for shipping and dispensing when needed, such as in a medical office.

FIG. 7 is a longitudinal cross section of tongue depressing device 10, which further illustrates the S shape of the device, which results in ends 11 and 12 being inversely oriented to one another. In certain embodiments, tongue depressing device 10 is a solid device. However, this is not a necessary feature of the device of the present invention and, in specific embodiments, tongue depressing device 10 has a hollow core which extends from end 11 to end 12 of the device or is limited to portions of the device. For example, the top surface 15 and the bottom surface 16 may be connected with one another only along the edges of tongue depressing device 10 (along the entire perimeter or edge, e.g., at ends 11 and 12 and sides 13 and 14) and have a hollow space between them. Alternatively, there may be sections of tongue depressing device 10 that are solid and other sections in which there are hollow spaces between the top surface 15 and the bottom surface 16. Less material, such as food grade plastic, is needed in such embodiments which, as a result are particularly environmentally friendly.

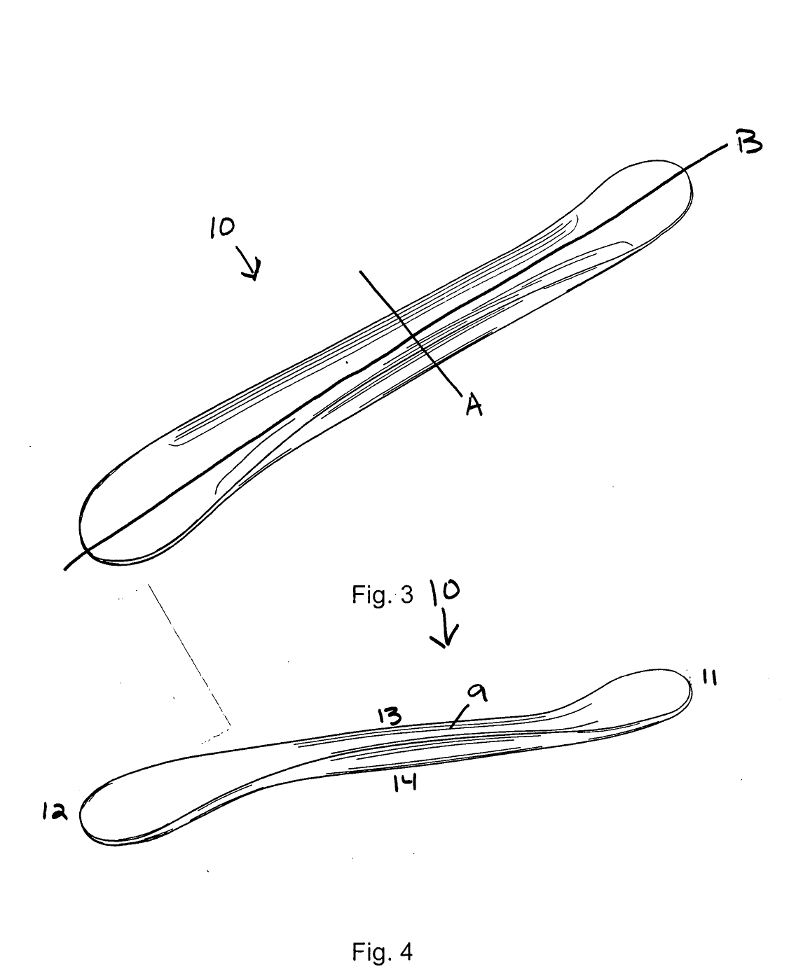

FIGS. 3 and 4 represent further perspectives of tongue depressing device 10 and illustrate its channeled configuration and curved surfaces on sides 13 and 14. The channeled configuration comprises a slight depression formed by the curved surfaces on sides 13 and 14. The depth of the depression can be varied as needed. In specific embodiments, on the convex surface, the channel extends to the midpoint of the tongue depressing device 10; beyond that point on the surface is flat or substantially flat. For example, in FIG. 3, on top surface 15, the channel extends to midpoint A. In alternative embodiments, the tongue depressing device 10 is an elongated member substantially in a plane having a first end and a second end and a top surface and a bottom surface, wherein the first end curves away from the plane toward the top surface and the second end curves away the plane toward the bottom surface and the top surface near the first end substantially conforms to the contour and shape of the tongue of an individual.

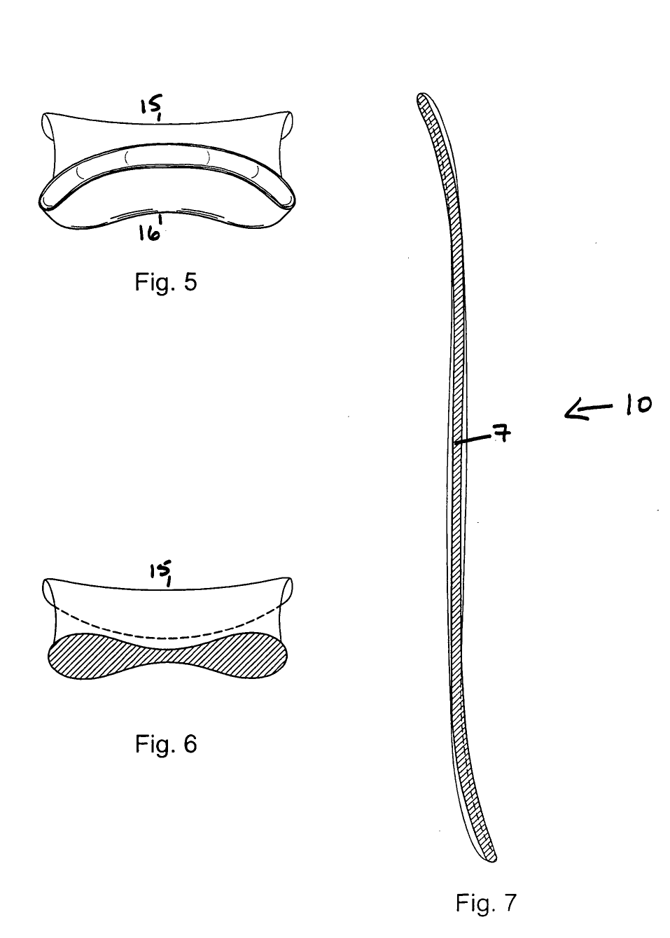

FIG. 5 is a view of tongue depressing device 10 from end 1 1 or 12, showing upper surface 15 and lower surface 16.

FIG. 6 is a transverse cross section, showing upper surface 15 and cross section 17. In this embodiment, the tongue depressing device 10 is not of constant thickness; the thickness varies from side 13 to side 14.

Tongue depressing device 10 is made from a non-toxic plastic suitable for introduction into the mouth, such as any food grade, FDA approved or FDA approvable plastic. It can be made by a variety of processes, such as an injection molding process, extrusion process or other process by which the desired configuration can be achieved. Whatever material and process are used, tongue depressing device 10 is preferably molded or otherwise formed as a single piece.

As mentioned above, there are numerous aspects of the tongue depressing device of the present invention which make it particularly useful and user friendly, from the perspective of both the individual being examined and the health care professional. For example, as a result of its ergonomic configuration and concomitant ability to conform to the shape of the tongue in both the lengthwise and crosswise dimensions, tongue depressing device 10 is easily inserted into the mouth, feels more comfortable while in position than the often used wooden tongue depressor and interferes less with the ability of the user to see into the mouth and throat. Also as a result of its design, which includes two flat ends (rounded or not rounded), tongue depressing device 10 has a “handle” at each end.

An additional aspect of the present invention which makes it useful in many contexts is the wide variety of features that can be included simply and in a cost effective way. For example, the surface can be smooth throughout or can be smooth in some areas and textured, somewhat rough or otherwise uneven in others. In some embodiments, selected sections can be textured, somewhat rough or otherwise uneven to increase the ability of the user to immobilize or control the movement of an individual's tongue or to obtain samples of cells or fluids from the oral cavity. Further, the surface can bear markings (e.g., units of linear measurement) which make it possible to use the device as a measuring instrument. Such markings can be present on either or both ends, along a side or sides or both and must be present in such a location that they can be seen by the user. In those embodiments in which such markings are present on both ends, for example, they are typically present on the convex surface of both ends (e.g., on surface 16 near rounded end 11 and on surface 15 near rounded end 12 of FIG. 2) so that either end can be used for measuring. In those embodiments in which such markings are present along either or both sides, they are placed in such a manner that the user can determine the size of lesions or other characteristics of the tongue or inner surface of the cheek. For example, such markings can be placed on top surface 15 (e.g., on the left side of top surface 15), bottom surface 16 (e.g., the right side of bottom surface 16)or both. Such markings can also be placed on tongue depressing device 10 in such a manner that lesions in the beginning or top of the throat and across the tongue can be measured. They can be placed, for example, on surface 16 near or at end 11, on surface 15 at or near end 12 or both. Markings are present on surface 15, surface 16 or both in such a manner that when tongue depressing device 10 is place in the mouth, the limits are visible to the user.

Flavorings, coloring agents and/or scents can also be included in tongue depressing device 10. For example, a flavoring such as a fruit, candy or bubble gum flavor, can be incorporated into the device or coated onto the device to produce a device that will be particularly appealing to children. For adults, coffee, chocolate or mocha flavorings might be of use. Coloring agents, which increase the eye appeal of tongue depressing device 10 and can also serve to distract children while they are being examined, can also be incorporated into or coated onto the surface of the device. Similarly, designs or other graphics, which can be educational or merely for entertainment, can be features of tongue depressing device 10.

It is often difficult to see with ease into an individual's mouth during examination, in part because of the lack of light. In one embodiment of this invention, tongue depressing device 10 bears at least two compartments (at least a first compartment and a second compartment) which are separated by a membrane which prevents mixing of the contents of the two compartments. The first compartment contains a substance which, when mixed with a substance in the second compartment, produces a temporary light source (e.g., by a chemical reaction). When the membrane is disrupted, the two substances combine and a temporary light source is produced. The light is not emitted until the contents of the compartments interact, such as when a user snaps the device in order to rupture the membrane. The temporary light source makes it easier for the user to view the mouth and/or throat of the individual being examined. In a further embodiment, a material that absorbs light is used to produce a temporary light source. This material can be in a compartment on the surface of tongue depressing device 10 or the device is impregnated with, has incorporated within or is coated with a light absorbing material. Alternatively, tongue depressing device 10 can comprise a LED and low power source paper film battery that, when squeezed or otherwise subjected to sufficient pressure, generates light sufficient to illuminate the mouth and/or throat.

While the invention has been described in detail and pictorially shown in the accompanying drawings, it is not to be limited to such details, since many changes and modifications may be made to the invention without departing from the spirit and scope thereof. Other embodiments will be evident to those skilled in the art and are within the scope of the present invention and the following claims.

1. A tongue depressing device which is an S-shaped device comprising a first end, an elongated midsection and a second end, wherein the first end and the second end each have a curved terminal portion and the curved terminal portion of the first end and the curved terminal portion of the second end project away from the elongated midsection in opposite directions and wherein the tongue depressing device conforms to the transverse contour and the longitudinal shape of the tongue of an individual.

2. The tongue depressing device of claim 1 wherein the midsection is channeled and which is composed of a rigid material with sufficient resilience to conform to the cross sectional contour and the elongated shape of the tongue of an individual.

3. The tongue depressing device of claim 1 which is composed of food grade material.

4. The tongue depressing device of claim 3 wherein the food grade material is food grade plastic.

5. The tongue depressing device of claim 2, wherein the first end and the second end are rounded.

6. The tongue depressing device of claim 5 which is composed of food grade plastic.

7. The tongue depressing device of claim 6 which bears text, medically related shapes, designs or patterns on the midsection.

8. The tongue depressing device of claim 1 which bears on a surface at least a first compartment and a second compartment which are separated by a membrane, wherein the first compartment contains a substance which when mixed with a substance in the second compartment produces a temporary light source.

9. The tongue depressing device of claim 4, wherein at least a portion of the surface of the elongated midsection is textured.

10. The tongue depressing device of claim 4 wherein at least one surface bears measuring units.

11. An S-shaped tongue depressing device which comprises a first end an elongated channeled midsection and a second end and which is ergonomically designed in such a manner that the first end or the second end can be introduced into the mouth to depress the tongue and in such a manner that it conforms to the transverse contour and the longitudinal shape of the tongue of an individual, thereby reducing the extent to which visualization of the mouth and throat are blocked by the tongue depressing device in the mouth of the individual.

12. The S-shaped tongue depressing device of claim 11 which is composed of food-grade material.

13. The S-shaped tongue depressing device of claim 12 wherein the food-grade material is food-grade plastic.

14. The S-shaped tongue depressing device of claim 11, wherein the first end and the second end are rounded.

15. The tongue depressing device of claim 11 which is composed of food-grade plastic.

16. The tongue depressing device of claim 15 which bears text on the midsection.

17. The tongue depressing device of claim 13 which bears on a surface at least a first compartment and a second compartment which are separated by a membrane, wherein the first compartment contains a substance which when mixed with a substance in the second compartment produces a temporary light source.

18. The tongue depressing device of claim 13, wherein at least a portion of the surface of the elongated midsection is textured.

19. The tongue depressing device of claim 15, wherein at least one surface bears measuring units.

20. A tongue depressing device comprising
an elongated member, substantially in a plane, having a first end and a second end, and
a top surface and a bottom surface, wherein
the first end curves away from the plane toward the top surface and the second end curves away from the plane toward the bottom, and
the top surface near the first end substantially conforms to the contour and shape of a tongue of an individual.

21. The tongue depressing device of claim 20, wherein the elongated member is channeled and which is composed of a rigid material with sufficient resilience to conform to the cross sectional contour and the elongated shape of the tongue of an individual.
